(19) United States
(12) Patent Application Publication
(10) Pub. No.: US 2006/0286165 A1
(43) Pub. Date: Dec. 21, 2006

(54) Taste-masked pharmaceutical particle, the preparation and use thereof

(76) Inventors: Jilong Ge, Changzhou City (CN); Yongrui Tu, Changzhou City (CN); Xudong Ge, Chanzhou City (CN); Jian'an Liu, Changzhou City (CN)

Correspondence Address: Hong J. Xu, Suite 330, 3975 University Drive, Fairfax, VA, 22030, US

(21) Appl. No.: 11/240,997

(22) Filed: Sep. 30, 2005

(30) Foreign Application Priority Data
Jun. 20, 2005 (CN) CN200510078609.1

(51) Int. Cl. A61K 9/20

(52) U.S. Cl. 424/464

(57) ABSTRACT

The present invention provides a taste-masked pharmaceutical particle prepared by a polymer blending process, a process for preparing the particle, and the use of the particle in preparing the orally disintegrating tablets. In this process, a micronized active drug ingredient is mixed with three kinds of pharmaceutically acceptable polymer adjuvants to yield a complete and uniformly mixed sheet with smooth surface and good plasticity. The sheet is knocked into small blocks, and then pulverized and pelleted to yield a taste-masked pharmaceutical particle whose bitter or peculiar taste is not felt in 120 s. The taste-masked particle can release the drug in a very high speed in an acidic solution (pH=1.0) and release the drug in a high speed in a solution with low acidity (pH=4.0), and effectively disperse in a solution with very low acidity (pH=6.8). The taste-masked particle can be directly tabulated after mixing with appropriate adjuvant to yield an orally disintegrating tablet with a uniform content, low friability, and ability to disintegrate in about 45 s.

### FIELD OF THE INVENTION

The present invention relates to a taste-masked pharmaceutical particle prepared by a polymer blending process, a process for preparing the particle, and the use of the particle in preparing the orally disintegrating tablets.

### BACKGROUND OF THE INVENTION

In order to further improve the pliability of the patients in taking drugs, the research on the orally disintegrating tablets is very active recently and the market product is continuously increasing. The orally disintegrating tablets can completely disintegrate in the oral cavity in several seconds and then the drug is taken into the stomach by swallowing saliva several times, then the drug is absorbed in the gastrointestinal tract. Direct tabulating is a commonly used process for preparing the orally disintegrating tablets. This process has a high productivity and low production cost, and the produced orally disintegrating tablets can completely disintegrate in about 30 s. A key technical problem to be solved in this process is how to eliminate the bitter or peculiar taste of the drug. The bitter or peculiar taste of some drugs cannot disappear in a long time even a small amount of the drug is remained in the oral cavity.

Anti-schizophrenia drugs such as risperidone, olanzapine, aripiprazole, and antianxietic drugs such as lorazepam, diazepam, and estazolam are especially suitable for being formulated into orally disintegrating tablets to enhance the pliability of the special patients suffering from schizophrenia or anxiety in taking drugs. However, these active drug ingredients have a bitter or peculiar taste, even a small amount in the oral cavity can make people feel unpleasant bitter or peculiar taste that is not easy to disappear.

There are a variety of processes for eliminating the bitter or peculiar taste of these drugs. One simple and early used process is to add a flavoring agent such as menthol, lemon oil, etc into the tablet formula, or increase the amount of the sweetening agent used, but this process does not work for some active drug ingredients with heavier bitter or peculiar taste. Another process is directly coating the drug or coating the particles containing the drug and appropriate adjuvant to mask the bitter or peculiar taste of the drug. This process is the most commonly used for masking the taste of drugs presently.

U.S. Pat. No. 5,215,755 discloses pelletizing ibuprofen, polyvinylpyrrolidone, and sodium laurylsulfate on a fluidizer with water, coating the prepared dry particle with an aqueous solution of hydroxyethyl cellulose HEC and hydroxypropylmethyl cellulose HPMC to yield a taste-masked ibuprofen particle with a weight gain of 18%. The particle is mixed with appropriate adjuvant and tabulated to yield the chewing tablets. This literature does not assess the effect of masking effect on ibuprofen. In view of that hydroxyethyl cellulose and hydroxypropylmethyl cellulose are both water-soluble materials, it is rather difficult to attain a good masking effect.

Someone dissolves water-insoluble cellulose acetate CA and polyvinylpyrrolidone PVP in a mixed solvent of acetone and alcohol and uses the solution to coat the pharmaceutical particle for the purpose of taste masking. U.S. Pat. No. 4,851,226 and U.S. Pat. No. 5,876,759 disclose the preparation of the taste-masked particle of p-acetylaminophenol by using this process. And the particle is used to prepare the chewing tablets and orally disintegrating tablets. The PVP and CA are used as a coating material, and the release of the drug in the stomach is slowed down or even stopped if the ratio of CA:PVP used is great, while if the ratio of CA:PVP used is less, a good masking effect in the oral cavity is not easy to achieve.

In order to accelerate the release of the drug from the coated taste-masked particle in the gastric juice, someone uses a polymer coating material sensitive to gastric acid, a copolymer of dimethylaminoethyl methacrylate-neutral methacrylate (Eudragit NE 100), or adds small molecular acid-sensitive substances to the coating material. U.S. Pat. No. 6,221,402 discloses coating particle containing sildenafil citrate with three layers of coats of Eudragit NE 30D, Eudragit E 100, and sugar, respectively. The obtained coated particle is effectively taste-masked, but the process is too complicated. U.S. Pat. No. 6,586,012 discloses coating the particle of levofloxaxin with the solution of cellulose acetate CA and a copolymer of dimethylaminoethyl methacrylate-neutral methacrylate (Eudragit E 100) in acetone, with a weight gain of 111%. The obtained coated particle releases very little drug in a buffer solution of pH=7.6, but releases the drug very fast in an acid solution of pH=1.2. U.S. Pat. No. 6,740,341 discloses coating the particle of dextrometrophan with a mixture of cellulose acetate CA, a copolymer of dimethylaminoethyl methacrylate-neutral methacrylate (Eudragit E 100) and talc to yield the taste masked particle, with a weight gain of 30%. U.S. Pat. No. 6,551,617 discloses a process which not only realizes the effective taste masking but also enhances the releasing rate of the drug in an acidic solution by adding a small amount, say, about 3% of triethanolamine into the coating polymer material to yield taste-masked particle of acetylaminophenol and caffeine. There is one shortcoming when an acid sensitive material is used to coat the particle for taste masking, i.e., a small number of patients with low level of gastric acid can only absorb little drug or even cannot absorb the drug.

Taste-masked drug powder can also be prepared by mixing a drug with appropriate adjuvant and then spray drying. U.S. Pat. No. 6,197,348 discloses an isopropanol-dispersed system of risperidone and Eudragit RS 100, yielding a taste-masked risperidone powder after spray drying. U.S. Pat. No. 4,835,188 discloses a water-dispersed system of a mixture of ibuprofen, ethyl cellulose, and dibutyl sebacate, yielding taste-masked ibuprofen powder after spray drying. The drug powder prepared by spry drying has a loose structure and is difficult to mask the taste of the drugs with heavier bitter or peculiar taste.

### BRIEF DESCRIPTION OF THE DRAWINGS

FIG. 1 is the DSC Analysis to the polymer blending particle obtained in Example 1 analyzed by differential scanning calorimetry (DSC).

FIG. 2 is the DSC Analysis to the simple mixture of the three kinds of polymer material (Eudragit E100, PVP K30, and PEG4000) in Example 1 analyzed by differential scanning calorimetry (DSC).

### DETAIED DESCRIPTION OF THE INVENTION

In order to overcome the shortcomings of the taste-masked pharmaceutical particle of the prior arts, the present invention provides a taste-masked pharmaceutical particle prepared by a polymer blending process, a process for preparing the particle, and the use of the particle in the preparation of the orally disintegrating tablets.

In particular, one aspect of the present invention relates to a taste-masked pharmaceutical particle prepared by a polymer blending process, the particle containing an active drug ingredient and three kinds of pharmaceutically acceptable polymer adjuvants

In a preferred embodiment, the active drug ingredient in the taste-masked pharmaceutical particle of the present invention denotes the drug having little solubility in water but certain solubility in dilute acid. Preferably, said active drug ingredient is selected from anti-schizophrenia drugs such as risperidone, olanzapine, aripiprazole, and antianxietic drugs such as lorazepam, diazepam, and estazolam. These drugs are used in the formulation as free base. Also preferably, the amount of these active drug ingredients is 10%-150% of the total weight of the mixture of the three kinds of polymer material, and the most preferred amount of these active drug ingredients is 50%-100% of the total weight of the mixture of the three kinds of polymer material.

In another preferred embodiment, said first pharmaceutically acceptable polymer adjuvant in the three kinds of pharmaceutically acceptable polymer adjuvant in the taste-masked pharmaceutical particle of the present invention is a pharmaceutically acceptable polymer material soluble in an acidic aqueous solution, which accounts for 10%-90% of the total weight of the three polymer materials, most preferably 30%-70%. Preferably it is a copolymer of dimethylaminoethyl methacrylate-neutral methacrylate and/or polyvinylacetaldiethylamine acetate (AEA).

In another preferred embodiment, said second pharmaceutically acceptable polymer adjuvant in the three kinds of pharmaceutically acceptable polymer adjuvants in the taste-masked pharmaceutical particle of the present invention is a solid pharmaceutically acceptable polymer material easily soluble in water, which accounts for 5%-60% of the total weight of the three polymer materials, most preferably 15%-40%. It is preferably polyvinylpyrrolidone (PVP).

In another preferred embodiment, said third pharmaceutically acceptable polymer adjuvant in the three kinds of pharmaceutically acceptable polymer adjuvants in the taste-masked pharmaceutical particle of the present invention is a polymer material soluble in water, which accounts for 5%-35% of the total weight of the three polymer materials, most preferably 10%-25%. It is preferably polyethylene glycol (PEG); the block copolymer of oxyethylene-oxypropylene (poloxamer).

In another preferred embodiment, said third pharmaceutically acceptable polymer adjuvant in the three kinds of pharmaceutically acceptable polymer adjuvants in the taste-masked pharmaceutical particle of the present invention is a pharmaceutically acceptable polymer material with a melting point of 25° C.-100° C., which accounts for 5%-35% of the total weight of the three polymer materials, most preferably 10%-25%.

In another preferred embodiment, said third pharmaceutically acceptable polymer adjuvant in the three kinds of pharmaceutically acceptable polymer adjuvants in the taste-masked pharmaceutical particle of the present invention is a polymer material with plasticization function and antiadhesive performance, which accounts for 5%-35% of the total weight of the three polymer materials, most preferably 10%-25%.

Preferably, said third pharmaceutically acceptable polymer adjuvant in the taste-masked pharmaceutical particle of the present invention is polyethylene glycol and/or a block copolymer of oxyethylene-oxypropylene.

Another aspect of the present invention relates to a process for the preparation of said taste-masked pharmaceutical particle. That is, mixing three kinds of pharmaceutically acceptable polymer materials to yield a polymer blending with peculiar performance according to the idea borrowed from the process commonly used in the study of the modification of polymer materials—polymer blending. A drug-containing particle can be prepared by adding the drug with a bitter or peculiar taste to the polymer materials while doing the polymer blending.

The process for preparing the taste-masked pharmaceutical particle by polymer blending is as follows:

Step 1. The active drug ingredient and the three kinds of pharmaceutically acceptable polymer adjuvants are pulverized, and the active drug ingredient and the first pharmaceutically acceptable polymer adjuvant are sieved through 200 mesh, while the second and third pharmaceutically acceptable polymer adjuvants are sieved through 100 mesh.

Step 2. The active drug ingredient and the three kinds of pharmaceutically acceptable polymer adjuvants are dry mixed in a certain ratio to yield a uniformly mixed dry powder, and an adequate amount of wetting agent was sprayed onto the dry powder in a high speed stirred mixer in two portions to yield the wet polymer mixture.

Step 3. The wet soft material is rolled in a biaxial roller to yield an incomplete sheet, which is divided into two parts and piled up into two layers. The two layers were rolled in the biaxial roller for the second time to yield a more complete sheet, which is divided into two parts and piled up into two layers. The two layers are rolled in the biaxial roller for the third time to yield an even more complete sheet. Such a procedure is repeated for 5-20 times or more to yield a complete sheet with smooth surface, uniform mixing, and good flexibility.

Step 4. The wet sheet is dried at a certain temperature.

Step 5. The sheet is knocked into small blocks, pulverized and sieved to yield a particle of 60-200 mesh. An appropriate amount of glidant, 1-10% of silica gel fine powder is added to the particle, and the mixture is rounded in a centrifugal baller at 50-90° C. for 15-30 min. Fine powder is removed with a sieve of 200 mesh to yield the taste-masked pharmaceutical particle.

It is preferable in step 2 that the active drug ingredient is micronized to give the particle with a diameter of about 5 μm.

In a preferred embodiment of the above process, the wetting agent is purified water or a mixture containing ethanol and purified water and the content of ethanol is not more than 30% by volume.

In another preferred embodiment of the above process, the amount of the wetting agent used is 30%-40% by weight of the dry powder.

In another preferred embodiment of the above process, the temperature used for drying the wet sheet is 50-100° C.

In another preferred embodiment of the above process, the glidant is silica gel fine powder.

In another preferred embodiment of the above process, the amount of the glidant used is 1%-10% by weight of the particle.

A further aspect of the present invention relates to the use of the taste-masked pharmaceutical particle in the preparation of the orally disintegrating tablets. The taste-masked pharmaceutical particle prepared by the process possesses good compressibility and can be directly tabulated to prepare the orally disintegrating tablets after mixing with appropriate adjuvant. The particle possesses good flowability and the tablet obtained by direct tableting possesses good content uniformity.

Analysis of the Particle:

Differential scanning calorimetry (DSC) is used to analyze the polymer blending particle and the result shows that in the temperature range of 40° C.-60° C., there is no Δ H peak of phase transition, indicating that the third kind of polymer material has already been dissolved into polyvinylpyrrolidone.

The third kind of polymer material (polyethylene glycol or a block copolymer of oxyethylene with oxypropylene) and the second kind of polymer material polyvinylpyrrolidone are dissolved into each other by multiple extrusions with water as a wetting agent, wherein the first kind of polymer material (Eudragit E 100 powder or AEEA powder) is uniformly dispersed. The surface of the particles of the first kind of polymer material is partly dissolved in the wetting agent through multiple extrusions with a mixture of water and ethanol; and after drying, the first kind of polymer material is uniformly dispersed in the mixture of the second kind of polymer material and the third kind of polymer material, and the first kind of polymer material becomes into a partly continuous phase. Because the first kind of polymer material exists as a partly continuous phase and it is hard to dissolve into a neutral aqueous solution, the polymer blending material dissolved into the saliva slowly and can be used in taste masking of a drug. Because the first kind of polymer material can rapidly dissolve into an acidic aqueous solution, the second kind of polymer material easily dissolves into water, and the third kind of polymer material is soluble in water, the polymer blending material can rapidly dissolve into an aqueous solution with low acidity (pH=4).

Differential scanning calorimetry (DSC) is used to analyze the taste-masked pharmaceutical particle and the result shows that the Δ H peak corresponding to the melting course of the active drug ingredient is apparent, and the phase of the drug active ingredient does not change, indicating that the process of the present invention does not greatly affect the stability and absorbability of the drug.

The process of the present invention for preparing the taste-masked pharmaceutical particle is very convenient and the product yield is high. The bitter or peculiar taste of the taste-masked pharmaceutical particle is not felt in the oral cavity within 120 s. The particle can rapidly release the drug in an acidic solution (pH=1.0) or a solution with lower acidity (pH=4), meaning that the release of the drug in the human body depends little on the gastric acid, and low gastric acid level will not affect the absorption to the drug.

The taste-masked pharmaceutical particle prepared by the process of the present invention is tasted by 6 volunteers and no bitter taste is felt within 120 s. The taste-masked pharmaceutical particle can rapidly release the drug in a medium of pH≦4.0. The taste-masked pharmaceutical particle can also release the drug completely in a medium of pH≧4.0, but this will take a longer time and depends on the particular pH value.

The taste-masked pharmaceutical particle possesses good compressibility and flowability, and good orally disintegrating tablets can be achieved after mixing with appropriate adjuvant and being tabulated. The disintegration time of the said tablets is less than 45 s. The content uniformity meets the requirement of USP26, and the friability measured according to the method of American pharmacopeia is <0.4%.

### EXAMPLES

The materials used in the examples are as follows:

A pharmaceutically acceptable polymer material soluble in an acidic aqueous solution: a copolymer of dimethylaminoethyl methacrylate-neutral methacrylate, Eudragit E100 (produced by Rohm Co., Germany).

A pharmaceutically acceptable polymer material easily dissolved in water:polyvinylpyrrolidone, PVP K30 (produced by Basf Co., Germany).

A water-soluble pharmaceutically acceptable polymer material with a low melting point: polyethylene glycol PEG4000 (produced by SASOL Co., Germany); a block copolymer of oxyethylene and oxypropylene, Poloxamer F68 (produced by Basf Co., Germany).

Active drug ingredients: risperidone, olanzapine, aripiprazole, estazolam, diazepam, and lorazepam.

glidant: silica gel fine powder N-20 (produced by Wacker Co., Germany).

### Example 1

Preparation of the Polymer Blending Particle (1)

Eudragit E100 was pulverized and sieved through 200 mesh, and polyethylene glycol PVP K-30 and PEG4000 were pulverized and sieved through 100 mesh. The sieved Eudragit E100, PVP K-30, and PEG4000 were weighed according to the ratio in Table 1, fed to a high speed stirred mixer with a rotational speed of 1000 rpm, and mixed for 10 min. Under stirring, a wetting agent, purified water, was sprayed in two portions and the mixture was stirred to mix for another 10 mins to yield a wet polymer blending particle. The wet particle was rolled in a biaxial roller for the first time to yield an incomplete sheet and the incomplete sheet was divided into two parts and piled up into two layers, which were rolled in the biaxial roller for the second time to yield a more complete sheet. The more complete sheet was divided into two parts and piled up into two layers, which were rolled in the biaxial roller for the third time to yield an even more complete sheet. Such a procedure was repeated, and after 10 times of rolling, a soft sheet with smooth appearance, uniformity, and good flexibility was obtained. The soft sheet was dried at 60° C. to yield a sheet with good plasticity. The sheet was knocked into small blocks, pulverized and sieved to yield 370 g of particle of 60-200 mesh with a yield of 86%. The fine powder passing through 200 mesh accounted for only 14%. 8 g of silica gel (N-20) fine powder was added to the particle of 60-200 mesh, which was rounded in a centrifugal baller at 60° C. for 30 min. Fine powder was removed with a sieve of 200 mesh to yield 357 g of the polymer blending particle.

TABLE 1
Raw materials for preparing polymer blending
particle (1) and their amount

| | | | |
|---|---|---|---|
| No. | Name | Amount | Ratio |

| | | | | |
|---|---|---|---|---|
| 1 | Copolymer of dimethylaminoethyl | 200 | g | 46% |
| | methacrylate-neutral methacrylate, | | | |
| | Eudragit E100 | | | |
| 2 | Polyvinylpyrrolidone, PVP K-30 | 150 | g | 35% |
| 3 | Polyethylene glycol, PEG4000 | 80 | g | 19% |
| 4 | Purified water | 135 | mL | — |

Analysis of the Obtained Polymer Blending Particle (1) by Differential Scanning Calorimetry (DSC)

Experimental Conditions:

Temperature range for scanning: 35° C.-200° C., heating-up rate: 10° C./min, sample weight: about 12 mg.

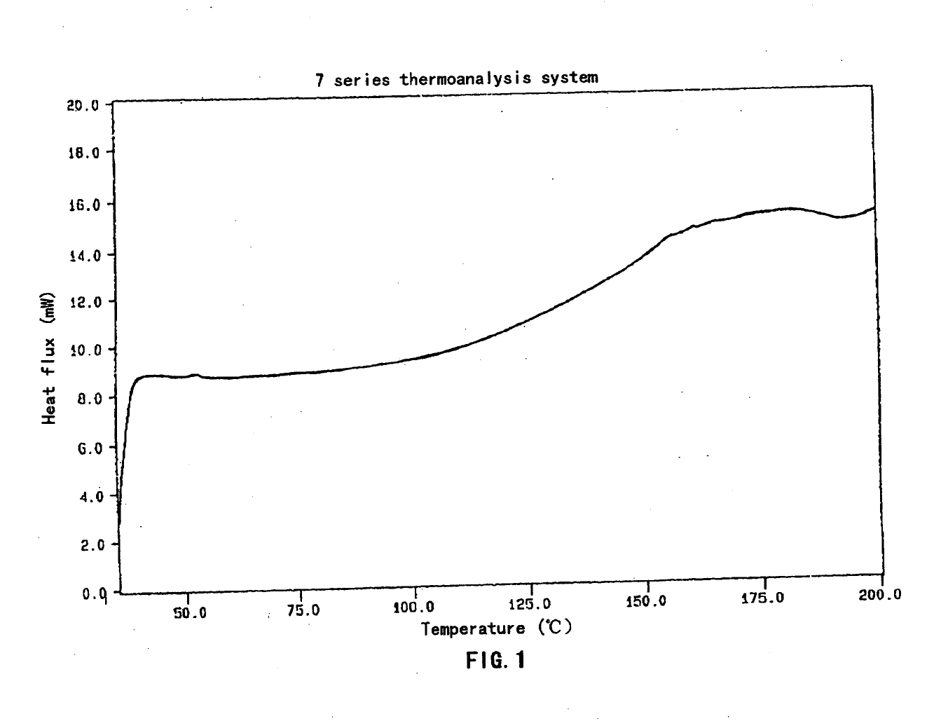

The DSC pattern is shown in FIG. 1.

The result demonstrated that there was no apparent phase transition heat effect in the whole temperature range for scanning, 35° C.-200° C., indicating that polyethylene glycol PEG4000 and PVP K-30 had been dissolve into each other.

Eudragit E100, PVP K-30, and PEG4000 were simply mixed according to the ratio shown in Table 1, and the simple mixture was analyzed using the same differential scanning calorimetry (DSC) and experimental conditions.

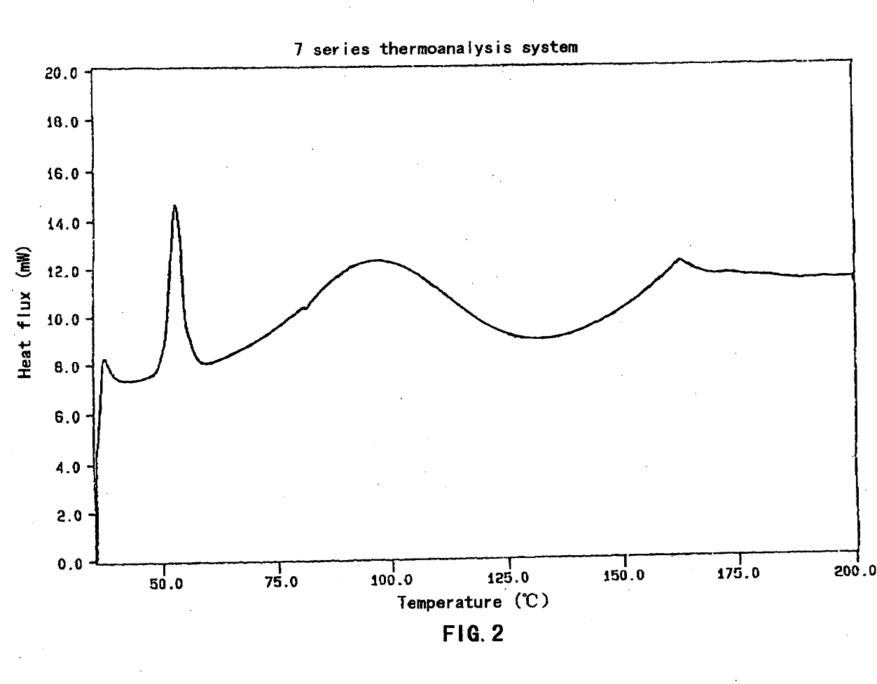

The DSC figure is shown in FIG. 2.

The result demonstrated that there was a strong endothermic peak near 46° C., indicating the existence of the phase of polyethylene glycol.

### Example 2

Preparation of the Polymer Blending Particle (2)

Eudragit E100 was pulverized and sieved through 200 mesh, and PVP K-30 and Poloxamer F68 were pulverized and sieved through 100 mesh. The sieved Eudragit E100, PVP K-30, and Poloxamer F68 were weighed according to the ratio in Table 2, fed to a high speed stirred mixer with a rotational speed of 1000 rpm, and mixed for 10 min. Under stirring, a wetting agent, ethanol-containing purified water (purified water/ethanol=80/20), was sprayed in two portions and the mixture was stirred to mix for another 10 min to yield a wet polymer blending particle. The particle was rolled in a biaxial roller for the first time to yield an incomplete sheet and the incomplete sheet was divided into two parts and piled up into two layers, which were rolled in the biaxial roller for the second time to yield a more complete sheet. The more complete sheet was divided into two parts and piled up into two layers, which were rolled in the biaxial roller for the third time to yield an even more complete sheet. Such a procedure was repeated, and after 10 times of rolling, a soft sheet with smooth appearance, uniformity, and good flexibility was obtained. The soft sheet was dried at 90° C. to yield a sheet with good plasticity. The sheet was knocked into small blocks, pulverized and sieved to yield 386 g of particle of 60-200 mesh with a yield of 90%. 8 g of silica gel (N-20) fine powder was added to the particle of 60-200 mesh, which was rounded in a centrifugal baller at 60° C. for 30 min. Fine powder was removed with a sieve of 200 mesh to yield 372 g of the polymer blending particle.

TABLE 2
Raw materials for preparing polymer blending
particle (2) and their amount

| No. | Name | Amount | | Ratio |
|---|---|---|---|---|
| 1 | Copolymer of dimethylaminoethyl | 200 | g | 46% |
| | methacrylate-neutral methacrylate, | | | |
| | Eudragit E100 | | | |
| 2 | Polyvinylpyrrolidone PVP K-30 | 150 | g | 35% |
| 3 | Block copolymer of | 80 | g | 19% |
| | oxyethylene-oxypropylene, Poloxamer F68 | | | |
| 4 | Purified water + ethanol (V/V = 80/20) | 130 | mL | — |

### Example 3

Preparation of the Taste-Masked Risperidone Particle

The pretreatment of Eudragit E100 and PVP K-30 was the same as that in Example 1.

Poloxamer F68 was pulverized and sieved through 100 mesh.

Drug powder of Risperidone with a diameter of about 5 μm was prepared by gas stream pulverization.

Using the same process as described in Example 1, the sieved Eudragit E100, PVP K-30, Poloxamer F68, and risperidone pharmaceutical particle were weighed according to the ratio in Table 3, mixed, made into a wet particle with ethanol-containing purified water as a solvent (purified water/ethanol=80/20), and rolled in a biaxial roller for 15 times to yield a risperidone-containing soft sheet with smooth appearance, uniformity, and good flexibility.

The risperidone-containing sheet was dried at 90° C.

The dried sheet was knocked into small blocks, pulverized and sieved to yield 261 g of risperidone-containing particle of 60-200 mesh with a yield of 78%.

6 g of silica gel (N-20) fine powder was added to the risperidone-containing particle, which was rounded in a centrifugal bailer at 60° C. for 30 min. Fine powder was removed with a sieve of 200 mesh to yield 253 g of taste-masked risperidone particle.

TABLE 3
Raw materials for preparing taste-masked
risperidone particle and their amount

| No. | Name | Amount | | Ratio |
|---|---|---|---|---|
| 1 | Risperidone | 120 | g | 36% |
| 2 | Copolymer of dimethylaminoethyl | 100 | g | 30% |
| | methacrylate-neutral methacrylate, | | | |
| | Eudragit E100 | | | |
| 3 | Polyvinylpyrrolidone PVP K-30 | 75 | g | 22% |
| 4 | Block copolymer of | 40 | g | 12% |
| | oxyethylene-oxypropylene, Poloxamer F68 | | | |
| 5 | Purified water + ethanol (V/V = 80/20) | 115 | mL | — |

### Example 4

Preparation of the Taste-Masked Olanzapine Particle

The pretreatment of Eudragit E100, PVP K-30, and PEG4000 was the same as that in Example 1.

Olanzapine drug powder with a diameter of about 5 μm was prepared by gas stream pulverization.

Using the same process as described in Example 1, the sieved Eudragit E100, PEG4000, PVP K-30, and olanzapine drug powder were weighed according to the ratio in Table 4, mixed, made into a wet particle with ethanol-containing purified water (purified water/ethanol=90/10) as a solvent, and rolled in a biaxial roller for 15 times to yield an olanzapine-containing soft sheet with smooth appearance, uniformity, and good flexibility.

The olanzapine-containing sheet was dried at 90° C.

The dried sheet was knocked into small blocks, pulverized, and sieved to yield 275 g of olanzapine-containing particle of 60-200 mesh with a yield of 82%.

6 g of silica gel (N-20) fine powder was added to the olanzapine-containing particle, which was rounded in a centrifugal baller at 60° C. for 30 min. Fine powder was removed with a sieve of 200 mesh to yield 261 g of the taste-masked olanzapine particle.

TABLE 4
Raw materials for preparing taste-masked
olanzapine particle and their amount

| No. | Name | Amount | | Ratio |
|---|---|---|---|---|
| 1 | Olanzapine | 120 | g | 36% |
| 2 | Copolymer of dimethylaminoethyl | 100 | g | 30% |
| | methacrylate-neutral methacrylate, | | | |
| | Eudragit E100 | | | |
| 3 | Polyvinylpyrrolidone, PVP K-30 | 75 | g | 22% |
| 4 | Polyethylene glycol PEG4000 | 40 | g | 12% |
| 5 | Purified water + ethanol (V/V = 90/10) | 120 | mL | — |

### Example 5

Preparation of the Taste-Masked Lorazepam Particle

The pretreatment of Eudragit E100 and PVP K-30 was the same as that in Example 1.

Poloxamer F68 was pulverized and sieved through 100 mesh

Lorazepam drug powder with a diameter of about 5 μm was prepared by gas stream pulverization.

Using the same process as described in Example 1, the sieved Eudragit E100, PVP K-30, Poloxamer F68, and lorazepam drug powder were weighed according to the ratio in Table 5, mixed, made into a wet particle with ethanol-containing purified water as a solvent (purified water/ethanol=90/10), and rolled in a biaxial roller for 15 times to yield a lorazepam-containing soft sheet with smooth appearance, uniformity, and good flexibility.

The lorazepam-containing sheet was dried at 90° C.

The dried sheet was knocked into small blocks, pulverized and sieved to yield 295 g of lorazepam-containing particle of 60-200 mesh with a yield of 88%.

6 g of silica gel (N-20) fine powder was added to the lorazepam-containing particle, which was rounded in a centrifugal baller at 90° C. for 15 min. Fine powder was removed with a sieve of 200 mesh to yield 275 g of taste-masked lorazepam particle.

TABLE 5
Raw materials for preparing taste-masked
lorazepam particle and their amount

| No. | Name | Amount | | Ratio |
|---|---|---|---|---|
| 1 | Lorazepam | 120 | g | 36% |
| 2 | Copolymer of dimethylaminoethyl | 100 | g | 30% |
| | methacrylate-neutral methacrylate, | | | |
| | Eudragit E100 | | | |
| 3 | Polyvinylpyrrolidone, PVP K-30 | 75 | g | 22% |
| 4 | Block copolymer of | 40 | g | 12% |
| | oxyethylene-oxypropylene, Poloxamer F68 | | | |
| 5 | Purified water + ethanol (V/V = 90/10) | 120 | mL | — |

### Examples 6-8

Preparation of the Taste-Masked Particles of Aripiprazole, Estazolam, and Diazepam

The pretreatment of Eudragit E100, PVP K-30, and Poloxamer F68 was the same as that in Example 2.

Drug powders of aripiprazole, estazolam, and diazepam with a diameter of about 5 μm were prepared respectively by gas stream pulverization.

Using the same process as described in Example 2, the sieved Eudragit E100, PVP K-30, Poloxamer F68, and drug powder were mixed according to the amount of various materials in Table 6 and subjected to various operations. The amount of the three taste-masked pharmaceutical particle is also shown in Table 6.

TABLE 6
Raw materials for preparing taste-masked particle of aripiprazole,
estazolam, and diazepam and their amount

| | | Example | | Example | | Example | |
|---|---|---|---|---|---|---|---|
| No. | Name | 6 | | 7 | | 8 | |
| 1 | Aripiprazole | 120 | g | — | | — | |
| 2 | Estazolam | — | | 120 | g | — | |
| 3 | Diazepam | — | | — | | 120 | g |
| 4 | Copolymer of | 100 | g | 100 | g | 100 | g |
| | dimethylaminoethyl | | | | | | |
| | methacrylate-neutral | | | | | | |
| | methacrylate, Eudragit E100 | | | | | | |
| 5 | Polyvinylpyrrolidone, | 75 | g | 75 | g | 75 | g |
| | PVP K-30 | | | | | | |
| 6 | Block copolymer of | 40 | g | 40 | g | 40 | g |
| | oxyethylene-oxypropylene, | | | | | | |
| | Poloxamer F68 | | | | | | |
| 7 | Purified water + ethanol | 115 | mL | 115 | mL | 115 | mL |
| | (V/V = 80/20) | | | | | | |
| 8 | Amount of prepared | 269 | g | 277 | g | 290 | g |
| | taste-masked particle | | | | | | |

### Example 9

Evaluation of the Taste-Masked Particle

(1) Evaluation of the Taste Masking Effect

The taste-masked particles of risperidone, olanzapine, lorazepam, aripiprazole, estazolam, and diazepam were tasted by 6 healthy volunteers, respectively. 50-100 mg of taste-masked pharmaceutical particle was put into the oral cavity of each person each time. The pharmaceutical particle was spit out as soon as an apparent bitter or peculiar taste was savored and the time was recorded. The test results are shown in Table 7.

TABLE 7
The time when the 6 healthy volunteers savored a bitter or peculiar taste
in tasting various taste-masked pharmaceutical particles (unit: s)

| | Taste-masked | Taste-masked | Taste-masked | Taste-masked | Taste-masked | Taste-masked |
|---|---|---|---|---|---|---|
| | risperidone | olanzapine | lorazepam | aripiprazole | estazolam | diazepam |
| No. of | particle/ | particle/ | particle/ | particle/ | particle/ | particle/ |
| volunteer | 50 mg | 100 mg | 50 mg | 100 mg | 50 mg | 100 mg |
| 1 | 139 | 164 | 210 | 173 | 249 | 226 |
| 2 | 132 | 149 | 194 | 192 | 287 | 208 |
| 3 | 145 | 182 | 182 | 160 | 268 | 196 |
| 4 | 150 | 177 | 207 | 157 | 242 | 243 |
| 5 | 123 | 163 | 193 | 186 | 302 | 230 |
| 6 | 169 | 152 | 165 | 167 | 274 | 211 |
| Average | 143 | 165 | 192 | 173 | 270 | 219 |

The test result shows that the bitter or peculiar taste of all the 6 taste-masked pharmaceutical particles was not savored within 120 s.

(2) The Release of the Taste-Masked Pharmaceutical Particle Under Different pH

According to the method for measuring the dissolution of the tablet in Chinese pharmacopoeia, 20 mg of taste-masked particle was put into 900 mL of medium and the dissolution was observed under 37° C. and 50 rpm.

a. Medium of pH=1.0: 9 mL of concentrated hydrochloric acid was diluted to 1000 mL with purified water.

b. Medium of pH=4.0: 0.1 mol/L of acetate buffer solution

c. Medium of pH=6.8: 0.05 mol/L of phosphate buffer solution

TABLE 8
The release of the taste-masked pharmaceutical particle under different pH (unit: s)

| | Polymer | | | | | | | | | | | | | |
|---|---|---|---|---|---|---|---|---|---|---|---|---|---|---|
| | mixture | | Risperidone | | Olanzapine | | Lorazepam | | Aripiprazole | | Estazolam | | Diazepam | |
| | particle | | taste-masked | | taste-masked | | taste-masked | | taste-masked | | taste-masked | | taste-masked | |
| pH | (2) | | particle | | particle | | particle | | particle | | particle | | particle | |
| 1.0 | 42 | s/cd^{a} | 27 | s/cd | 32 | s/cd | 40 | s/cd | 37 | s/cd | 39 | s/cd | 35 | s/cd |
| 4.0 | 153 | s/cd | 177 | s/cd | 203 | s/cd | 182 | s/cd | 229 | s/cd | 193 | s/cd | 261 | s/cd |
| 6.8 | 521 | s/dis^{b} | 638 | s/dis | 797 | s/dis | 553 | s/dis | 980 | s/dis | 600 | s/dis | 1300 | s/dis |

^{a}Completely dissolved
^{b}Dispersed

The test result demonstrates that the polymer blending particle containing no drug and the taste-masked particle containing drugs can rapidly dissolve and release drug in the medium of pH=1.0 (equivalent to the human gastric juice); with the increase of pH, the dissolution of the particle becomes more and more difficult. In the medium of pH=6.8 (equivalent to the human intestinal juice), the particle can not dissolve completely, but can disperse completely.

(3) The Effect of the Taste-Masked Pharmaceutical Particle in the Preparation of the Orally Disintegrating Tablets

a. Formulation of the Orally Disintegrating Tablets

As shown in Table 9

TABLE 9
formulation of the orally disintegrating tablets

| Ingredients | Unit dosage (mg) | Batch number |
|---|---|---|
| Taste-masked pharmaceutical | 10 | 2000 tablets |
| particle | | |
| Mannitol | 96 | |
| Cross-linked | 9 | |
| polyvinylpyrrolidone | | |
| Low substituted | 5 | |
| hydroxypropylcellulose | | |
| Microcrystalline cellulose | 25 | |
| Aspartam | 2.0 | |
| Menthol | 0.5 | |
| Magnesium stearate | 1.0 | |
| Silica gel fine powder | 1.5 | |
| Sum | 150 | |

b. Preparation Procedure

Mannitol, cross-linked polyvinylpyrrolidone, Low substituted hydroxypropylcellulose, and microcrystalline cellulose were sieved through 100 mesh, respectively, and fully mixed with the taste-masked particle, aspartam, and menthol, and magnesium stearate and silica gel fine powder were added. The stirring was continued for 5 min, the mixture was tabulated. The weight of each of the tablet was controlled to about 150 mg, and the hardness was about 30 N.

c. The Analysis Result on the Quality of the 6 Kinds of Orally Disintegrating Tablets

As shown in Table 10

TABLE 10
The analysis result of the 6 kinds of orally disintegrating tablets

| | | Risperidone | Olanzapine | Lorazepam | Aripiprazole | Estazolam | Diazepam |
|---|---|---|---|---|---|---|---|
| | | orally | orally | orally | orally | orally | orally |
| | Measuring | disintegrating | disintegrating | disintegrating | disintegrating | disintegrating | disintegrating |
| Items | method | tablets | tablets | tablets | tablets | tablets | tablets |
| Disintegrating | See Note 1 | 32 s | 37 s | 29 s | 43 s | 39 s | 31 s |
| time | | n = 6 | n = 6 | n = 6 | n = 6 | n = 6 | n = 6 |
| Content | See Note 2 | RSD = 1.1% | RSD = 0.8% | RSD = 1.4% | RSD = 1.2% | RSD = 0.9% | RSD = 1.2% |
| uniformity | | | | | | | |
| Friability | American | 0.21% | 0.17% | 0.38% | 0.10% | 0.13% | 0.29% |
| | pharmacopeia | | | | | | |

Note 1:
The time required for 6 preparations to completely disperse was measured respectively in 2 mL of unstirred water at 37° C., and the average value was calculated.
Note 2:
The content of 10 preparations was measured respectively and the RSD was calculated.

The result in Table 10 shows that the orally disintegrating tablets prepared by the taste-masked pharmaceutical particle possess uniform content and low friability, and can completely disintegrate in 45 s, indicating that the taste-masked pharmaceutical particle possesses good flowability and compressibility.

1. A taste-masked pharmaceutical particle, which contains an active drug ingredient and three kinds of pharmaceutically acceptable polymer adjuvants.

2. The taste-masked pharmaceutical particle of claim 1, wherein the weight of said active drug ingredient is about 10%-150% by weight of the three kinds of pharmaceutically acceptable polymer adjuvants.

3. The taste-masked pharmaceutical particle of claim 1, wherein said active drug ingredient is a drug having low solubility in water but certain solubility in dilute acid.

4. The taste-masked pharmaceutical particle of claim 1, wherein the first kind of pharmaceutically acceptable polymer adjuvant in said three kinds of pharmaceutically acceptable polymer adjuvants is a pharmaceutically acceptable polymer material soluble in an acidic aqueous solution, which accounts for about 10%-90% by weight of the three kinds of pharmaceutically acceptable polymer adjuvants.

5. The taste-masked pharmaceutical particle of claim 1, wherein the second pharmaceutically acceptable polymer adjuvant in said three kinds of pharmaceutically acceptable polymer adjuvants is a water-soluble solid pharmaceutically acceptable polymer material, which accounts for about 5%-60% by weight of the three kinds of pharmaceutically acceptable polymer adjuvants.

6. The taste-masked pharmaceutical particle of claim 1, wherein the third pharmaceutically acceptable polymer adjuvant in said three kinds of pharmaceutically acceptable polymer adjuvants is a water-soluble polymer material, which accounts for about 5%-35% by weight of the three kinds of pharmaceutically acceptable polymer adjuvants.

7. The taste-masked pharmaceutical particle of claim 1, wherein the said third pharmaceutically acceptable polymer adjuvant is a polymer material with a melting point in the range of 25° C.-100° C., which accounts for about 5%-35% by weight of the three kinds of pharmaceutically acceptable polymer adjuvants.

8. The taste-masked pharmaceutical particle of claim 1, wherein the said third pharmaceutically acceptable polymer adjuvant is a polymer material with plasticization function and antiadhesive performance, which accounts for about 5%-35% by weight of the three kinds of pharmaceutically acceptable polymer adjuvants.

9. The taste-masked pharmaceutical particle of claim 1, wherein said active drug ingredient is selected from anti-schizophrenia drugs such as risperidone, olanzapine, aripiprazole, and antianxietic drugs such as lorazepam, diazepam, and estazolam.

10. The taste-masked pharmaceutical particle of claim 1, wherein said first pharmaceutically acceptable polymer adjuvant is a copolymer of dimethylaminoethyl methacrylate-neutral methacrylate.

11. The taste-masked pharmaceutical particle of claim 1, wherein said second pharmaceutically acceptable polymer adjuvant is polyvinylpyrrolidone.

12. The taste-masked pharmaceutical particle of claim 1, wherein said third pharmaceutically acceptable polymer adjuvant is polyethylene glycol and/or a block copolymer of oxyethylene with oxypropylene.

13. A process for preparing the taste-masked pharmaceutical particle of claim 1, comprising:
pulverizing an active drug ingredient and three kinds of pharmaceutically acceptable polymer adjuvants, and sieving the active drug ingredient and the first pharmaceutically acceptable polymer adjuvant through 200 mesh, while sieving the second and third pharmaceutically acceptable polymer adjuvants through 100 mesh;
mix-drying the active drug ingredient and the three kinds of pharmaceutically acceptable polymer adjuvants in a certain ratio to yield a uniformly mixed dry powder, and spraying an appropriate amount of wetting agent onto the dry powder in a high speed stirred mixer in two portions to yield the wet polymer mixture;
rolling the wet soft material in a biaxial roller to yield an incomplete sheet, which is divided into two parts and piled up into two layers; the two layers are rolled in the biaxial roller for the second time to yield a more complete sheet, which is divided into two parts and piled up into two layers; the two layers are rolled in the biaxial roller for the third time to yield an even more complete sheet; such a procedure is repeated for 5-20 times or more to yield a complete sheet with smooth surface, uniform mixing, and good flexibility;
drying the wet sheet at a certain temperature; and
knocking the sheet into small blocks, pulverizing and sieving to yield a particle of 60-200 mesh; adding an appropriate amount of glidant, 1-10% of silica gel fine powder, and rounding the mixture in a centrifugal baller at 50-90° C. for 15-30 min; removing the fine powder with a sieve of 200 mesh to yield the taste-masked pharmaceutical particle.

14. The process of claim 13, wherein the wetting agent is purified water or a mixture containing ethanol and purified water, wherein the content of ethanol is not more than 30% by volume.

15. The process of claim 13, wherein the amount of the wetting agent is 30%-40% by weight of the dry powder.

16. The process of claim 13, wherein the temperature for drying the wet sheet is 50-100° C.

17. The process of claim 13, wherein the glidant is silica gel fine powder.

18. The process of claim 13, wherein the amount of the glidant is 1%-10% by weight of the particle.

19. A process of preparing an orally disintegrating tablet comprising adding a taste-masked pharmaceutical particle of claim 1 into a tablet formulation.
